(19) United States
(12) Patent Application Publication
(10) Pub. No.: US 2018/0021011 A1
(43) Pub. Date: Jan. 25, 2018

(54) ULTRASOUND PUNCTURING METHOD AND APPARATUS

(71) Applicant: WHALE IMAGING, INC., Waltham, MA (US)

(72) Inventors: Yi LI, Beijing (CN); Jian LI, Beijing (CN); Xingbai HE, Beijing (CN)

(73) Assignee: Whale Imaging, Inc., Waltham, MA (US)

(21) Appl. No.: 15/546,506

(22) PCT Filed: May 4, 2015
(86) PCT No.: PCT/IB2015/001107

§ 371 Date: Jul. 26, 2017

(51) Int. Cl. A61B 8/08; A61B 17/34; A61B 8/00

(52) U.S. Cl. CPC A61B 8/0841; A61B 8/587; A61B 8/42; A61B 8/4444; A61B 17/3403; A61B 2017/3413

(57) ABSTRACT

An ultrasonic puncture method including the following steps: mounting a probe beam direction indicator on a probe by an indicator holder, with the orientation of the probe beam direction indicator the same as that of the probe, and enabling a light beam emitted by the probe beam direction indicator to rotate in a plane perpendicular to a beam plane of the probe; rotating the probe beam direction indicator to allow the light beam to coincide with the beam plane of the probe, and locking the probe beam direction indicator; and placing the probe on the body surface, and inserting a puncture needle along the light beam after a focus is found. The method can solve the problem of having difficulty in accurately and rapidly displaying the puncture needle on an ultrasonogram in free-hand puncture of a superficial tissue or superficial organ at present. An ultrasonic puncture device is also disclosed.

### TECHNICAL FIELD

The invention relates to the ultrasonic puncture field, in particular to a method and a device to support ultrasonic superficial puncture.

### BACKGROUND

In the 1970s, Goldberg and Holm invented an ultrasonic puncture probe capable of displaying a puncture needle on an ultrasonogram, thus realizing ultrasound-based visual guided puncture, and accurately reaching focuses. With the development of technology, such puncture technologies as indirect ultrasonic guided puncture, ultrasonic puncture adapter guided puncture, ultrasonic guided free-hand puncture gradually emerge.

The indirect ultrasonic guided puncture is mainly used to mark sites, but not monitor the trends and positions of syringe needles in real time, and also used for biopsy of huge masses. The ultrasonic puncture adapter guided puncture can fix the relative position of a puncture needle and a probe, and has very high accuracy. However, the puncture needle cannot change the angle freely and cannot be applied to puncture of superficial organs due to the fixed relative position.

The ultrasonic guided free-hand puncture is mainly used for puncture of superficial organs and superficial tissues. During operation, various requirements are required, for example, the long axis of the puncture needle shall be kept parallel with the beam plane when the needle is inserted, and the included angle between the puncture needle and skin cannot be larger than 60°. Before the needle is inserted, a puncture locating point is pressed again with a finger, and the ultrasonogram is observed, then a body surface locating point and an inserting point are marked, and the ultrasonogram is observed to estimate the inserting angle. After the needle is inserted, the needle is inserted a little bit from an anesthesia position, then the inserted needle is slightly lifted., and an active point thereof on the ultrasonogram is observed.

As the position shall he adjusted repeatedly until the puncture needle is displayed on the ultrasonogram during the ultrasonic guided free-hand puncture, it is unlikely that the ultrasonic puncture adapter guided puncture is applicable to the ultrasonic guided free-hand puncture. In addition, this process is painful for patients, and the requirements for doctors' experience and skills are very high.

### SUMMARY

The purpose of the invention is to provide an ultrasonic puncture method capable of solving the problem of having difficulty in accurately and rapidly displaying the puncture needle on an ultrasonogram in free-hand puncture of a superficial tissue or superficial organ at present.

The technical problem to be solved, in the invention adopts the following technical solution; an ultrasonic puncture method, comprising the following steps:

S10, installing a probe beam direction indicator on a probe by an indicator holder, with the orientation of the probe beam direction indicator the same as that of the probe, and enabling a light beam emitted by the probe beam direction indicator to rotate in a plane perpendicular to a beam plane of the probe;

S20, rotating the probe beam direction indicator to allow the light beam emitted by the probe beam direction indicator to coincide with the beam plane of the probe, and locking the probe beam direction indicator by a locking member; and

S30, placing the probe on the body surface, and inserting a puncture needle along the light beam projected by the probe beam direction indicator after a focus is found.

Preferably, the light beam emitted by the probe beam direction indicator is a straight light beam.

Preferably, in the step S20, the probe beam direction indicator is calibrated by an ultrasound phantom to allow the light beam emitted by the probe beam direction indicator to coincide with the beam plane of the probe.

The invention has the following beneficial effects: in the invention, the probe beam direction indicator is fixed on the probe by the indicator holder, and the light beam emitted by the probe beam direction indicator is adjusted to coincide with the beam plane of the probe; the puncture needle is inserted along the light beam projected by the probe beam direction indicator to directly reach a focus in free-hand puncture of a superficial tissue or superficial organ, and the puncture needle is kept in the beam plane of the probe, thus the puncture needle can be accurately and rapidly displayed on the ultrasonogram using the method.

Another purpose of the invention is to provide an ultrasonic puncture device capable of solving the problem or having difficulty in accurately and rapidly displaying the puncture needle on an ultrasonogram in free-hand puncture of a superficial tissue or superficial organ at present.

The invention further provides an ultrasonic puncture device comprising an indicator holder and a probe beam direction indicator. The indicator holder is capable of being removably fixed on a probe, an indicator mounting hole is arranged on the indicator holder, the probe beam direction indicator is rotatably mounted in the indicator mounting hole to enable a light beam emitted by the probe beam direction indicator to rotate in a plane perpendicular to a beam plane of the probe, and a locking member is arranged on the indicator holder for locking the probe beam direction indicator.

Preferably, the locking member is a locking bolt, a threaded through hole is arranged on the hole wall of the indicator mounting hole, the locking bolt is mounted in the threaded through hole, and an inner end of the locking bolt can abut against the probe beam direction indicator.

Preferably, the probe beam direction indicator is a laser sensor.

Preferably, the indicator holder comprises a probe fixed part and an indicator fixed part, the indicator mounting hole is arranged on the indicator fixed pan, the probe fixed part comprises a front plate, a rear plate and a connecting bolt, both the left ends of the front plate and the rear plate are fixedly connected to the right end of the indicator fixed part, and both the right ends of the front plate and the rear plate are connected by the connecting bolt, a probe mounting hole is enclosed by the right end of the probe fixed part, the front plate, the connecting bolt and the rear plate, and the probe is mounted in the probe mounting hole.

The invention has the following beneficial effects: the ultrasonic puncture device of the invention is provided with the indicator holder and the probe beam direction indicator, the probe beam direction indicator is rotatably mounted in the indicator mounting hole, and can be adjusted in use to keep the light beam emitted thereby in the beam plane of the probe, and the light beam projected by the probe beam direction indicator can accurately guide puncture of the puncture needle, thus being capable of solving the problem of having difficulty in accurately and rapidly displaying the puncture needle on an ultrasonogram in free-hand puncture of a superficial tissue or superficial organ at present.

### BRIEF DESCRIPTION OF THE DRAWINGS

FIG. 1 is a perspective view of an ultrasonic puncture device of the invention;

FIG. 2 is a front view of an ultrasonic puncture device of the invention;

FIG. 3 is a left view of an ultrasonic puncture device of the invention; and

FIG. 4 is a top view of an ultrasonic puncture device of the invention.

Marks in the figures are as follows: 1—indicator holder; 11—probe fixed part:, 111—front plate; 112—rear plate; 113—connecting bolt; 2—indicator fixed part; 2—probe beam direction indicator; 3—probe; 4—locking member; 5—light beam.

### DETAILED DESCRIPTION

The technical solution of the invention will be further described in combination with examples and drawings.

### EXAMPLE 1

The example provides an ultrasonic puncture method, comprising the following steps:

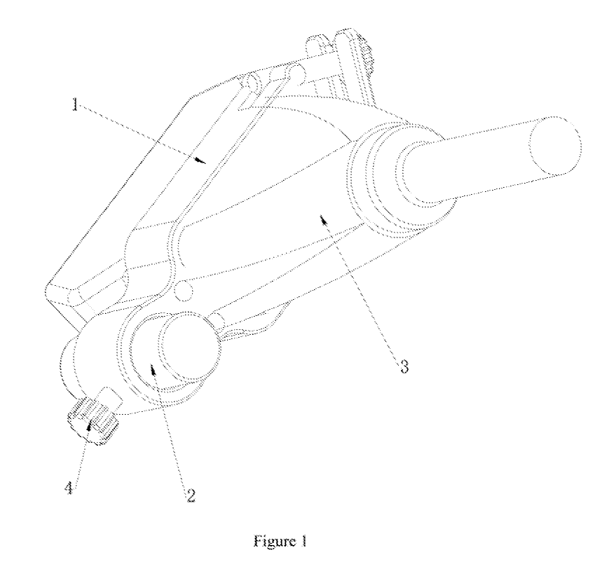

S10, mounting a probe beam direction indicator (2) on a probe (3) by an indicator holder (1), with the orientation of the probe beam direction indicator (2) the same as that of the probe (3), as shown in FIG. 1, both the probe beam direction indicator (2) and the probe (3) are arranged downward, and the probe beam direction indicator (2) is rotatably mounted on the indicator holder (1), thus enabling a light beam emitted by the probe beam direction indicator (2) to rotate in a plane perpendicular to a beam plane of the probe (3);

S20, rotating the probe beam direction indicator (2) to allow the light beam emitted by the probe beam direction indicator (2) to coincide with the beam plane of the probe (3), in the example, the probe beam direction indicator (2) is calibrated by an ultrasound phantom, after the probe beam direction indicator (2) is calibrated, the probe beam direction indicator (2) is locked by a locking member (4) to fix the relative position of the probe beam direction indicator (2) and probe (3);

S30, placing the probe (3) on the body surface, and inserting a puncture needle along the light beam projected by the probe beam direction indicator (2) after a focus is found, thus directly reaching the focus, and being capable of keeping the puncture needle in the beam plane of the probe (3).

In the example, the light beam emitted by the probe beam direction indicator (2) can be a straight light beam, or light beam of other shapes such as a cross light beam, and the light beam can guide puncture of the puncture needle by the part if any in the beam plane of the probe.

### EXAMPLE 2

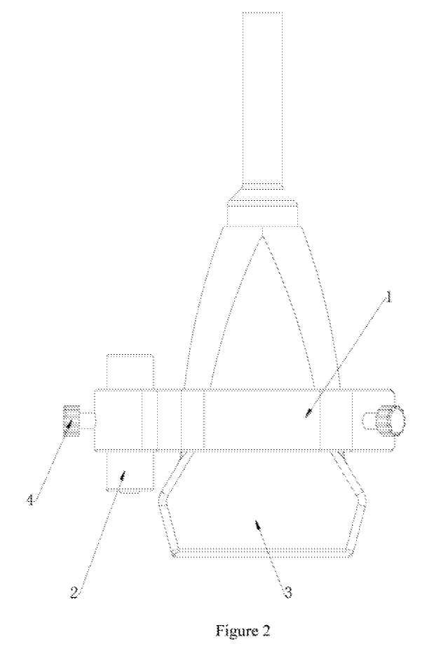

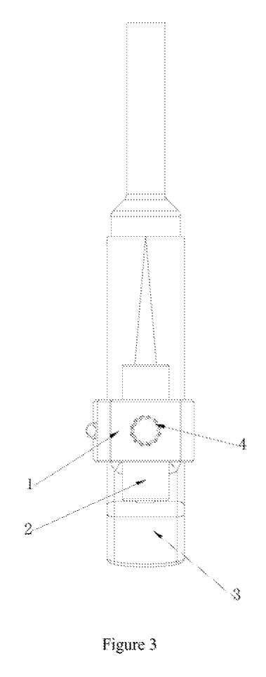

As shown in FIG. 1, FIG. 2 and FIG. 3, the example provides an ultrasonic puncture device comprising an indicator holder (1) and a probe beam direction indicator (2). The indicator holder (1) can be removably fixed on a probe (3). An indicator mounting hole is arranged on the indicator holder (1), the probe beam direction indicator (2) is rotatably mounted in the indicator mounting hole, both the probe beam direction indicator (2) and the probe (3) are arranged downward, enabling a light beam emitted by the probe beam direction indicator (2) to rotate in a plane perpendicular to a beam plane of the probe (3), and a locking member (4) is arranged on the indicator holder (1) for locking the probe beam direction indicator (2). In the example, the probe beam direction indicator (2) is a laser indicator capable of emitting straight light beams or light beams of various shapes such as cross light beams.

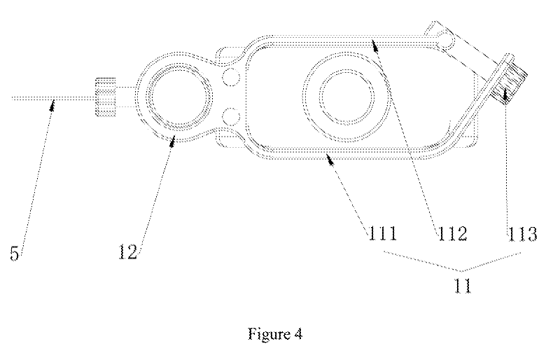

As shown in FIG. 4, the indicator holder (1) comprises a probe fixed part (11) and an indicator fixed part (12). An indicator mounting hole is arranged on the indicator fixed part (12). The probe fixed part (11) comprises a front plate (111), a rear plate (112) and a connecting bolt (113), both the left ends of the front plate (111) and the rear plate (112) are fixedly connected to the right end of the indicator fixed part (12), and both the right ends of the front plate (111) and the rear plate (112) are connected by the connecting bolt (113). In the example, the indicator fixed part (12), the front plate (111) and the rear plate (112) are integrated. A probe mounting hole is enclosed by the right end of the probe fixed part (11), the front plate (111), the connecting bolt (113) and the rear plate (112), and has a shape basically the same as the cross section shape of the probe (3), and the probe (3) is mounted in the probe mounting hole. The indicator holder (1) can be fixedly connected with the probe (3) by mounting the probe (3) in the probe mounting hole, and tightening the connecting bolt (113).

In the example, the locking member (4) is a locking bolt, a threaded through hole is arranged on the hole wall of the indicator mounting hole, the locking bolt is mounted in the threaded through hole, and an inner end of the locking bolt can abut against the probe beam direction indicator (2).

When the ultrasonic puncture device adjusts the angle between the light beam emitted by the probe beam direction indicator (2) and the beam plane of the probe (3), the light beam (e.g., a straight light beam) emitted by the probe beam direction indicator (2) irradiates on the ultrasound phantom. The locking member (4) is loosened, then the probe beam direction indicator (2) is rotated to keep a light beam (5) emitted thereby in the ultrasonic plane of the probe (3), and the locking member (4) is fastened to abut against the probe beam direction indicator (2).

In the example, the locking member can be a snap ring fixedly arranged on the indicator fixed part, the snap ring comprises two half rings, one ends of the two half rings are fixedly connected, and the other ends are connected by a bolt or served as free ends. When the other ends are connected by a bolt, the inner diameter of the snap ring is changed by loosening or tightening the bolt, thus clamping the probe beam direction indicator; and when the other ends are served as free ends, the probe beam direction indicator is clamped by elastic deformation of the free ends themselves.

The sequence of the examples is only for convenient description, and does not represent the advantages and disadvantages of the examples.

Finally, it should be understood that the examples are only considered to be illustrative for the technical solution of the invention instead of limitation thereto. Although the invention is described in detail in combination with the above examples, those of ordinary skill in the art shall understood that they can modify the technical solutions in the above examples or perform equivalent replacement on some technical features, but such modifications or replacements shall not allow the nature of corresponding technical solutions to depart from spirit and scope of the technical solutions in all examples of the invention.

1. An ultrasonic puncture method, characterized by comprising the following steps:
S10, mounting a probe beam direction indicator on a probe by an indicator holder, with the orientation of the probe beam direction indicator the same as that of the probe, and enabling a light beam emitted by the probe beam direction indicator to rotate in a plane perpendicular to a beam plane of the probe;
S20, rotating the probe beam direction indicator to allow the light beam emitted by the probe beam direction indicator to coincide with the beam plane of the probe, and locking the probe beam direction indicator by a locking member; and
S30, placing the probe on the body surface, and inserting a puncture needle along the light beam projected by the probe beam direction indicator after a focus is found.

2. The ultrasonic puncture method of claim 1, characterized in that the light beam emitted by the probe beam direction indicator is a straight light beam.

3. The ultrasonic puncture method of claim 1, characterized in that in the step S20, the probe beam direction indicator is calibrated by an ultrasound phantom to allow the light beam emitted by the probe beam direction indicator to coincide with the beam plane of the probe.

4. An ultrasonic puncture device, characterized by comprising an indicator holder and a probe beam direction indicator, the indicator holder being capable of being removably fixed on a probe, an indicator mounting hole being arranged on the indicator holder, the probe beam direction indicator being rotatably mounted in the indicator mounting hole to enable a light beam emitted by the probe beam direction indicator to rotate in a plane perpendicular to a beam plane of the probe, and a locking member being arranged on the indicator holder for locking the probe beam direction indicator.

5. The ultrasonic puncture device of claim 4, characterized in that the locking member is a locking bolt, a threaded through hole is arranged on the hole wall of the indicator mounting hole, the locking bolt is mounted in the threaded through hole, and an inner end of the locking bolt can abut against the probe beam direction indicator.

6. The ultrasonic puncture device of claim 5, characterized in that the probe beam direction indicator is a laser indicator.

7. The ultrasonic puncture device of claim 6, characterized in that the indicator holder comprises a probe fixed part and an indicator fixed part, the indicator mounting hole is arranged on the indicator fixed part, the probe fixed part comprises a front plate, a rear plate and a connecting bolt, both the left ends of the front plate and the rear plate are fixedly connected to the right end of the indicator fixed part, and both the right ends of the front plate and the rear plate are connected by the connecting bolt, a probe mounting hole is enclosed by the right end of the probe fixed part, the front plate, the connecting bolt and the rear plate, and the probe is mounted in the probe mounting hole.
